(19) United States
(12) Patent Application Publication
(10) Pub. No.: US 2009/0198293 A1
(43) Pub. Date: Aug. 6, 2009

(54) Microtransponder Array for Implant

(76) Inventors: Lawrence Cauller, Plano, TX (US); Richard Weiner, Dallas, TX (US)

Correspondence Address: GROOVER & Associates, BOX 802889, DALLAS, TX, 75380-2889, US

(21) Appl. No.: 12/324,000

(22) Filed: Nov. 26, 2008

Related U.S. Application Data
(63) Continuation-in-part of application No. 10/741,136, filed on Dec. 19, 2003.
(60) Provisional application No. 60/990,278, filed on Nov. 26, 2007.
(60) Provisional application No. 61/079,004, filed on Jul. 8, 2008.
(60) Provisional application No. 61/088,774, filed on Aug. 14, 2008.

(51) Int. Cl. A61N 1/375

(52) U.S. Cl. 607/2

(57) ABSTRACT

A wireless microtransponder array constructed as a single structure of joined microtransponders. The microtransponders can be configured as a linear array strip with connective material in between. The microtransponders can also be entirely embedded within a strip of material, or joined by a single, common substrate structure.

### CROSS REFERENCE TO RELATED APPLICATIONS

This application claims priority from provisional patent application 61/079,004 filed Jul. 8, 2008 and 60/990,278, filed on Nov. 26, 2007, which is hereby incorporated by reference. It is a continuation in part of non-provisional application Ser. No. 10/741,136 filed Dec. 19, 2003, and application 61/088,774 filed Aug. 14, 2008, which are also hereby incorporated by reference. This application may be related to the present application, or may merely have some drawings and/or disclosure in common.

### BACKGROUND

The numerous innovative teachings of the present application will be described with particular reference to a number of embodiments, including presently preferred embodiments (by way of example, and not of limitation), as well as other embodiments.

### BRIEF DESCRIPTION OF THE DRAWINGS

The disclosed inventions will be described with reference to the accompanying drawings, which show important sample embodiments of the invention and which are incorporated in the specification hereof by reference, wherein:

FIG. 1 is a functional schematic of a complete microtransponder for sensing and/or stimulating neural activity consistent with the present innovations.

FIG. 2 is an illustration of a laminar spiral micro-coil used in the construction of a microtransponder platform for stimulating neural activity consistent with the present innovations.

FIG. 3 is an illustration of a laminar spiral micro-coil electroplated onto a substrate consistent with the present innovations.

FIG. 4 is an illustration of a circuit diagram for a wireless microtransponder designed for independent auto-triggering operation (asynchronous stimulation) consistent with the present innovations.

FIG. 5 presents several graphs that summarize how wireless microtransponder stimulus frequency, stimulus current peak amplitude and stimulus pulse duration varies under different device settings and external RF power input conditions consistent with the present innovations.

FIG. 6 is an illustration of a circuit diagram for a wireless microtransponder with an external trigger signal de-modulator element to synchronize the stimuli delivered with a plurality other wireless microtransponders consistent with the present innovations.

FIG. 7 is a chart that illustrates de-modulation of an external interrupt trigger signal by differential filtering consistent with the present innovations.

FIG. 8 presents several graphs that summarizes the results from tests of a wireless microtransponder (with an external interrupt trigger de-modulator element) under different device settings and external RF power intensity conditions consistent with the present innovations.

FIG. 9A is an illustration of a deployment of a plurality of wireless microtransponders distributed throughout subcutaneous vascular beds and terminal nerve fields consistent with the present innovations.

FIG. 9B is an illustration of a deployment of wireless microtransponders to enable coupling with deep microtransponder implants consistent with the present innovations.

FIG. 9C is an illustration of a deployment of wireless microtransponders to enable coupling with deep neural microtransponder implants consistent with the present innovations.

FIG. 10 is an illustration of how wireless microtransponders can be deployed using a beveled rectangular hypodermic needle consistent with the present innovations.

FIG. 10A is an illustration of the current innovation for deployment of joined microtransponders deployed using a beveled rectangular hypodermic needle.

FIG. 11 is an illustration of a fabrication sequence for spiral type wireless microtransponders consistent with the present innovations.

FIG. 12A shows a perspective view of the basic embodiment of an array.

FIG. 12B shows a side view of the basic embodiment of an array.

FIG. 12C shows an overhead view of the basic embodiment of an array.

FIG. 13A shows a perspective view of an array comprising exposed electrodes through windows in the array.

FIG. 13B shows a side view of an array comprising exposed electrodes through windows in the array.

FIG. 13C shows an overhead view of an array comprising exposed electrodes through windows in the array.

FIG. 14A shows a perspective view of an array comprising an ion permeable strip.

FIG. 14B shows a side view of an array comprising an ion permeable strip.

FIG. 14C shows an overhead view of an array comprising an ion permeable strip.

FIG. 15A shows a perspective view of a slotted array.

FIG. 15B shows a side view of the slotted array.

FIG. 15C shows an overhead view of the slotted array.

FIG. 16A shows a perspective view of an array surrounded by an enveloping matrix.

FIG. 16B shows a side view of an array surrounded by an enveloping matrix.

FIG. 16C shows an overhead view of an array surrounded by an enveloping matrix.

### DETAILED DESCRIPTION OF THE PREFERRED EMBODIMENTS

A variety of medical conditions involve disorders of the neurological system within the human body. Such conditions may include paralysis due to spinal cord injury, cerebral palsy, polio, sensory loss, sleep apnea, acute pain, and so forth. One characterizing feature of these disorders may be, for example, the inability of the brain to neurologically communicate with neurological systems dispersed throughout the body. This may be due to physical disconnections within the neurological system of the body, and/or to chemical imbalances that can alter the ability of the neurological system to receive and transmit electrical signals, such as those propagating between neurons.

Advances in the medical field have produced techniques aimed at restoring or rehabilitating neurological deficiencies leading to some of the above-mentioned conditions. However, such techniques are typically aimed at treating the central nervous system and, therefore, are quite invasive. These techniques include, for example, implanting devices, such as electrodes, into the brain and physically connecting those devices via wires to external systems adapted to send and receive signals to and from the implanted devices. While beneficial, the incorporation of foreign matter into the human body usually presents various physiological complications, including surgical wounds and infection, which render these techniques potentially very challenging to implement with a risk of dangerous complications.

For example, the size of the implanted devices and wires extending therefrom may reduce or substantially restrict patient movement. Moreover, inevitable patient movements may cause the implanted device to shift, resulting in patient discomfort and possibly leading to the inoperability of the implanted device. Consequently, corrective invasive surgical procedures may be needed to reposition the device within the body, thereby further increasing the risk of infection and other complications.

In addition, an implanted device typically requires a battery to operate, and if the device is to remain within the body for prolonged periods, the batteries will need to be replaced, requiring additional surgical procedures that can lead to more complications. Furthermore, certain applications require that the implanted devices be miniaturized to the greatest extent possible, so they can be precisely implanted within the human body or so that a cluster of them can be implanted within a small defined area.

Publication US20020198572 by Weiner, for example, describes an apparatus for providing subcutaneous electrical stimulation. This device is certainly beneficial, providing pain relief by stimulating peripheral nerves, thus avoiding surgical interventions that target the brain or central nervous system (CNS). However, the device is bulky and has wire leads connecting the power sources to the implanted electrode.

Techniques such as those described in U.S. Publication 20030212440 by Boveja and related patents avoid the problem of battery replacement in a biostimulator by using a magnetic transmitter coil (RF transmission coil) placed over the region of the body that contains the implanted electrodes. This coil receives power and command signals via inductive coupling to generate stimulation pulses to activate motor units. Since the device contains no battery, the electrical power is derived from the externally generated RF field in the transmitting coil. However, this device is specifically designed for stimulus of the vagus nerve, and is not generally applicable. Further, the disclosed device still possesses a significant implant component with leads connecting the electrodes (alongside the vagus nerve) to the implanted stimulus receiver (in the chest).

Another approach is followed in devices similar to those described in U.S. Publication 20030212440 by Boveja made under the trademark BIONR and currently in clinical trials for the treatment of urinary urge incontinence and headaches. The BION® units are fairly large, ranging about 2 mm×10 mm×2 mm (thickness), and much smaller embodiments are preferred for implantation. Furthermore, BION® units must be hermetically sealed in order to protect the coils from the damaging effects of water and other bodily fluids. Additionally, BION® units require relatively high levels of externally applied RF power (often >1 watt) to provide the greater stimulus currents necessary for their primary purpose to activate stimulate individual muscles or muscle groups.

U.S. Publication 20050137652 by Cauller et al. provides for small, wireless neural stimulators. In this disclosed device, a plurality of single channel electrodes interface with the cellular matter, thus allowing smaller devices to be used without sacrificing efficacy. Because the subcutaneous tissue conducts electrical signals, the small electrodes are able to provide sufficient signal for stimulating neurons, in spite of the devices' small size and distance from the nerve.

U.S. Publication 20060206162 by Wahlstrand et al. also describes a device capable of transcutaneous stimulations with an array of electrodes that are attached to the skin surface on the back of the neck. However, this device contains a battery within the housing and is still quite large.

VeriChip® is the first FDA-cleared human-implantable RFID microchip. About twice the length of a grain of rice the device is glass-encapsulated (to seal the internal components away from the body), and implanted above the triceps area of an individual's right arm. Once scanned at the proper frequency, the VeriChip® responds with a unique sixteen-digit number which can correlate the user to information stored on a database for identity verification, medical records access and other uses. The data is not encrypted, causing serious privacy concerns, and there is some evidence that the devices may cause cancer in mice.

The clinical function of an electrical device such as a microtransponder, cardiac pacemaker lead, neurostimulation lead, or other electrical lead depends upon the device being able to maintain intimate anatomical contact with the target tissue (typically nerve or muscle tissue). All foreign substances implanted in the body are subject to a foreign body response from the surrounding host tissues. The body recognizes the implant as foreign, which triggers an inflammatory response followed by encapsulation of the implant with fibrous connective tissue (or glial tissues—called gliosis—when in the central nervous system). Scarring (fibrosis or gliosis) can also result from trauma to the anatomical structures and tissue surrounding the implant during the implantation of the device. Lastly, fibrous encapsulation of the device can occur after a successful implantation if the device is manipulated (some patients continuously fiddle with a subdermal/subcutaneous implant) or irritated by daily activities of the patient.

When scarring occurs around the implanted device, the electrical characteristics of the electrode-tissue interface degrade and the device may fail to function in a clinically significant way. For example, it may require additional electrical current from the lead to overcome the extra resistance imposed by the intervening scar. One of the observed faults of the VeriChip® design is that since it integrated with the surrounding tissue, it requires surgeons to surgically remove perfectly good flesh.

There are advantages to using even smaller, reliable, wireless implantable devices and/or methods adapted to treat neural or other biological disorders and to address aforementioned shortcomings, which include easy implantation and removal.

An embodiment of a wireless microtransponder includes an array. The array can comprise a removable joined array of embedded and joined microtransponders, facilitating easy removal of the array with minimal surgical invasion. An implantable array can be more easily removed after an acute treatment, or also in the event of malfunction or patient paranoia. This invention can allow for simpler removal of the actual microtransponders. In some embodiments, the design can incorporate an array of strongly joined individual microtransponders, so a surgeon can access and remove the array rather than individual microtransponders.

The disclosed innovations, in various embodiments, provide one or more of at least the following advantages:
Small size allowing multiple stimuli within a small area.
Ease of implantation, as the array permits implantation using a needle.
Ease of removal, as the solid array of joined microtransponders can be more easily extracted compared to individual microtransponders.
Lessened invasive surgical procedures for implantation and removal.

The numerous innovative teachings of the present application will be described with particular reference to the presently preferred embodiment (by way of example, and not of limitation).

Various embodiments of the present invention are directed towards the miniaturization of minimally invasive wireless micro-implants termed “microtransponders,” which may be small enough to allow numerous independent microtransponders to be implanted under a square inch of skin for sensing a host of biological signals or stimulating a variety of tissue responses. The microtransponders can operate without implanted batteries or wires by receiving electromagnetic power from pliable coils placed on the surface of the overlying skin. The microtransponder design is based upon wireless technology Radio Frequency Identification Devices (RFIDs).

The present application discloses new approaches to methods and apparatuses for providing minimally invasive wireless microtransponders that can be subcutaneously implanted and configured to sense a host of biological signals and/or stimulate a variety of tissue responses. The microtransponders contain miniaturized micro-coils that are formed by utilizing novel fabrication methods and have simplified circuit designs that minimize the overall size of the microtransponders. The unprecedented miniaturization of minimally invasive biomedical implants made possible with this wireless microtransponder technology would enable novel forms of distributed stimulation or high resolution sensing using micro-implants so small that implantation densities of 100 per square inch of skin are feasible.

The simplicity of the microtransponders allows extreme miniaturization, permitting many microtransponders to be implanted into a given area, usually by relatively noninvasive injection techniques. The microtransponders are biologically compatible, thus avoiding the need to seal the devices (as with the VeriChip® and further contributing to small size. Many biologically compatible materials and coatings are known, such as gold, platinum, SU-8, Teflon®, polyglycerols, or hydrophilic polymers such as polyethylene glycol (PEG). Additionally, many materials can be made biologically compatible by passivating the surface to render it non-reactive. In some embodiments, the microtransponder may include an anti-migration coating, such as a porous polypropylene polymer, to prevent migration away from the implant site. However, experiments to date indicate that the uncoated devices do not migrate. The tiny devices float independently in the tissue, moving only as the tissue moves, thus minimizing tissue rejection and encapsulation and maximizing longevity and effectiveness.

Wireless RFID technology involves the near-field magnetic coupling between two simple coils tuned to resonate at the same frequency (or having a harmonic that matches a harmonic or the fundamental frequency of the other coil). Throughout this document, references to tuning two coils to the “same frequency” includes having the frequencies of coils match at fundamental and/or harmonic frequencies. Radio Frequency (RF) electromagnetic power applied to one of these coils generates a field in the space around that power coil. Electrical power can be induced remotely in any remote coil placed within that power field as long as the remote coil is properly tuned to resonate at the same frequency as the power coil.

A miniaturized spiral shaped micro-coil in the microtransponder optimized for near field induction can be used. The micro-coil includes a non-conducting substrate, a conducting coil, and a photoresist layer patterned over the conducting coil, with the micro-coil electroplated onto the non-conducting substrate. The micro-coil can be used to both receive and transmit wireless signals such as a wireless power or wireless data signal.

Power can be delivered externally using near field coupling to deliver electrostimulation via a PDA-like programmable controller that allows the user to control the electrical parameters as needed for a given physiological condition. Near field coupling means the external driver needs to be close to the microtransponder (e.g. about 1 cm away), but increased distance (up to a point) can be achieved by adding coils or increasing the size. Protection from interference with other external RF sources is achieved in part by the short distance between the power source and microtransponder, but use of a select frequency and an encrypted link between the external and internal systems further reduces the possibility of implant activation by foreign RF sources.

An auto-triggering wireless microtransponder can be used to provide asynchronous electro-stimulation. The microtransponder of this embodiment includes a resonator element, a rectifier element, a stimulus voltage element, a stimulus discharger element, and a conducting electrode. The microtransponder is configured to discharge an electrical stimulus with a repetition rate that is controlled by the intensity of the externally applied RF power field.

A wireless microtransponder with an external trigger signal de-modulator element can be used to provide synchronized electro-stimulation. The microtransponder of this embodiment includes a resonator element, a rectifier element, an external trigger demodulator element, a stimulus timer element, a stimulus driver element, and a conducting electrode. The external trigger demodulator element is configured to receive a trigger signal from an external radio frequency (RF) power field. The stimulus driver element is configured to discharge an electrical stimulus when the external trigger demodulator element receives the trigger signal.

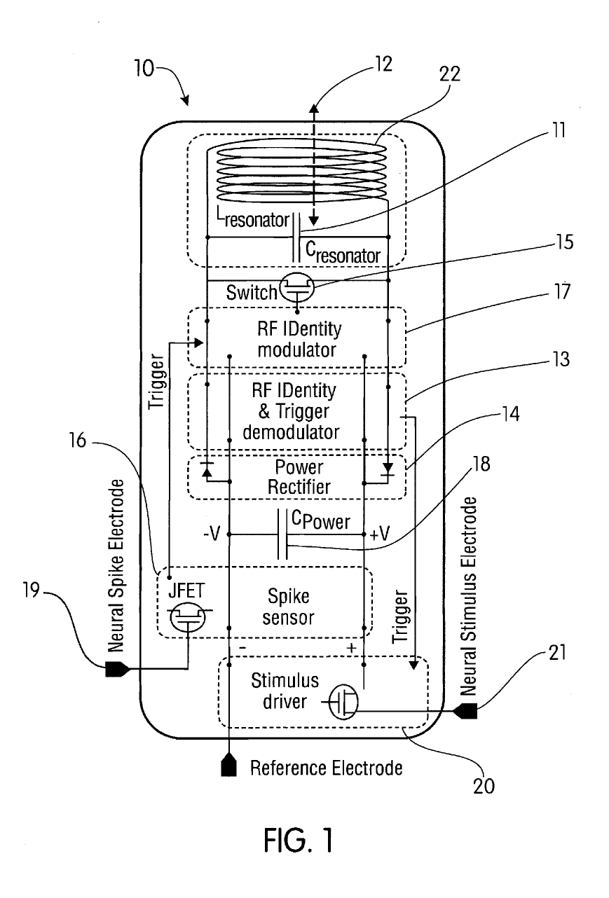

FIG. 1 is a functional schematic of a complete microtransponder for sensing and/or stimulating neural activity, in accordance with one embodiment. The circuit is designed for dependent triggering operation (synchronous stimulation). The circuit 10 includes electrical components adapted to electrically interface with neurons of peripheral nerves. The circuit 10 further includes electrical components which enable the microtransponder to wirelessly interact with systems external to the microtransponder. Such systems may include other transponders implanted within the body or external coils and/or a receiver. The wireless capabilities of the circuit 10 enable the delivery of electrical signals to and/or from the peripheral nerves. These include electrical signals indicative of neural spike signals and/or signals configured to stimulate peripheral nerves distributed throughout the subcutaneous tissue.

Accordingly, the circuit 10 includes the micro-coil 22 coiled about a central axis 12. The micro-coil 22 is coupled in parallel to a capacitor 11 and to an RF identity modulator 17 via a switch 15. The RF identity modulator 17 is coupled to an RF identity and trigger demodulator 13, which in turn is coupled to a rectifier 14. The rectifier 14 is coupled to a spike sensor trigger 16 and to a stimulus driver 20. The rectifier 14 and the spike sensor 16 are both coupled in parallel to a capacitor 18. In addition, the spike sensor 16 is coupled to a neural spike electrode 19, thereby electrically connecting the spike sensor 16 to neural transmission tissue (neurons). Similarly, the neural stimulus electrode 21 also connects the stimulus driver 20 to neural conduction tissue (axons). The spike sensor 16 is made up of one or more junction field effect transistors (JFET). As will be appreciated by those of ordinary skilled in the art, the JFET may include metal oxide semiconductors field effect transistors (MOSFETS).

The sensors, drivers, and other electronic components described in the present application can be fabricated using standard small scale or very large scale integration (VLSI) methods. Further, the spike sensor 16 is coupled to the RF identity modulator 17, which is adapted to modulate an incoming/carrier RF signal in response to neural spike signals detected by the spike sensor 16. In one embodiment, the neural electrodes (i.e., neural spike electrode 19 and neural stimulus electrode 21) to which the spike sensor 16 and the stimulus driver 20 are connected, respectively, can be bundled and configured to interface with neural conduction (axon) portion of a peripheral nerve.

One configuration of the above components, as depicted by FIG. 1, enables the microtransponder to operate as an autonomous wireless unit, capable of detecting spike signals generated by peripheral nerves, and relaying such signals to external receivers for further processing. It should be understood that the microtransponder performs such operations while being powered by external RF electromagnetic signals. The above-mentioned capabilities are facilitated by the fact that magnetic fields are not readily attenuated by human tissue. This enables the RF electromagnetic signals to sufficiently penetrate the human body so that signals can be received and/or transmitted by the microtransponder. In other words, the micro-coil 22 is designed and configured to magnetically interact with the RF field whose magnetic flux fluctuates within the space encompassed by the micro-coil 22. By virtue of being inductors, the micro-coils 22 convert the fluctuations of the magnetic flux of the external RF field into alternating electrical currents, flowing within the micro-coil 22 and the circuit 10. The alternating current is routed, for example, into the rectifier 14, which converts the alternating current into direct current. The direct current may then be used to charge the capacitor 18, thereby creating a potential difference across the JFET of the spike sensor 16.

In an exemplary embodiment, a gate of the spike sensor 16 JFET may be coupled via the neural spike electrode 19 to the neural transmission tissue (neurons). The gate of the spike sensor 16 JFET may be chosen to have a threshold voltage that is within a voltage range of those signals produced by the neural axons. In this manner, during spike phases of the neural axons, the gate of the spike sensor 16 becomes open, thereby closing the circuit 10. Once the circuit 10 closes, the external RF electromagnetic field generates an LC response in the coupled inductor 22 and capacitor 18, which then resonate with the external RF electromagnetic field, with its resonance matching the modulating frequency of the RF electromagnetic field. The LC characteristic of the circuit 10, as well as the threshold voltage of the gate of spike sensor 16 JFET, can be chosen to determine a unique modulation within the coupled micro-coil (i.e. inductor) 22 and capacitor 18, thereby providing a identifying signal for the microtransponder. Accordingly, the spike sensor 16 JFET provides the RF identity modulator 17 with a unique trigger signal for generating desired RF signals. The identity signal may indicate the nature of the neural activity in the vicinity of the microtransponder, as well as the location of the neural activity within the body as derived from the specific identified microtransponder position.

It should be appreciated that the RF capabilities, as discussed above with respect to the circuit 10, can render the microtransponder a passive device which reacts to incoming carrier RF signals. That is, the circuit 10 does not actively emit any signals, but rather reflects and/or scatters the electromagnetic signals of the carrier RF wave to provide signals having specific modulation. In so doing, the circuit 10 draws power from a carrier radio frequency (RF) wave to power the electrical components forming the circuit 10.

While the above-mentioned components illustrated in FIG. 1 may be used to receive signals from the microtransponder in response to spike signals generated by peripheral nerves, other components of circuit 10 of the microtransponder may include components for stimulating the peripheral nerves using the external RF signals. For example, the RF signals received by the micro-coil 22 may be converted to electrical signals, via the RF identity and trigger demodulator 13, so as to provide sufficient current and voltage for stimulating the peripheral nerves. Hence, the RF identity and trigger demodulator 13 derives power from an RF carrier signal for powering the stimulus driver 20, which delivers electrical signals suitable for stimulating neural conduction tissue (axons). This may be used to treat nerves that are damaged or that are otherwise physiologically deficient. Because of the nature of the identifying signal, a microtransponder can be selectively activated to provide electrostimulation.

It should be understood that, in certain embodiments, the minimum size for the microtransponders may be limited by the size of the micro-coil responsible for power induction, and secondarily by the size of the capacitors necessary for tuning power storage and timing. In fact, micro-coils less than 1 millimeter in diameter and just a few micrometers thick can provide sufficient wireless power to operate the complex micro-electronics that can be manufactured on integrated circuit chips that may be much smaller than these coils. Combining the sophisticated functionality of micro-electronic chips with the wireless performance of these micro-coils creates the smallest possible, minimally invasive implants, in the form of tiny flecks as small as 0.1 mm thick and 1 mm wide. The size and power advantages make it possible to add relatively complex digital electronics to the smallest transponder.

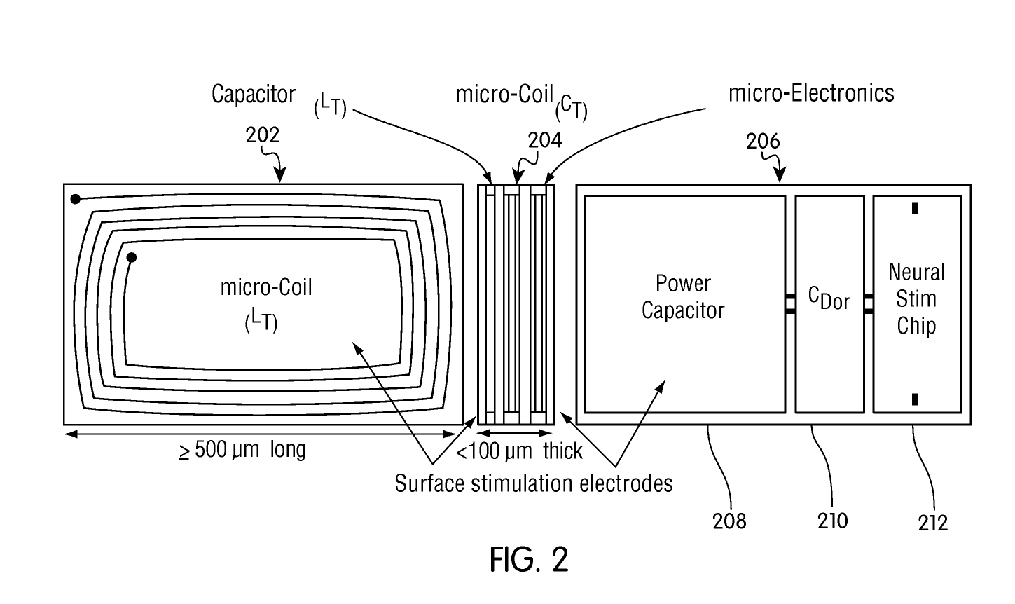

FIG. 2 is an illustration of a laminar spiral micro-coil power circuit used in the construction of a microtransponder platform for stimulating neural activity, in accordance with one embodiment. As depicted, herein, the mirotransponder includes a laminar spiral micro-coil (L_{T}) 202 coupled to a capacitor (C_{T}) 204, which in turn is coupled to a microelectronics chip 206. The microelectronics chip 206 includes a power capacitor element 208 coupled to a capacitor (C_{DUR}) element 210, which in turn is coupled to a neural stimulation chip element 212. In an exemplary embodiment of the microtransponder platform, the micro-coil is no more than 500 μm long by 500 μm wide and the combined thickness of the laminar spiral micro-coil (L_{T}) 202, capacitor (C_{T}) 204, and micro-electronics chip 206 is no more than 100 μm.

FIG. 3 is an illustration of a laminar spiral micro-coil electroplated onto a substrate, in accordance with one embodiment. As depicted in the drawing, conductor lines 302 are initially electroplated in a tight spiral pattern onto a non-reactive substrate (e.g., glass, silicon, etc.). In one embodiment, the laminar spiral micro-coil can include conductor lines 302 that are about 10 μm wide and the spacing 304 between the conductor lines 302 set at about 10 μm. In another embodiment, the laminar spiral micro-coil can include conductor lines 302 that are about 20 μm wide and the spacing 304 between the conductor lines 302 set at about 20 μm. It should be understood, however, that the widths of the conductor line 302 and line spacing 304 can be set to any value as long as the resulting micro-coil can produce the desired induced current for the desired application.

Platinum-iridium alloy is the preferred electroplating material to form the conductor lines 302. Gold or platinum are other acceptable conductors that can be utilized to form the conductor lines 302.

In certain embodiments, once the spiral micro-coil has been electroplated onto the substrate, a polymer-based layer is spun on top of the micro-coils to provide a layer of protection against corrosion and decay once implanted. Long-term studies of animals with SU-8 implants have verified the biocompatibility of SU-8 plastic by demonstrating that these SU-8 implants remain functional without signs of tissue reaction or material degradation for the duration of the studies. Therefore, typically, the polymer-based layer is comprised of an SU-8 or equivalent type of plastic having a thickness of approximately 30 μm.

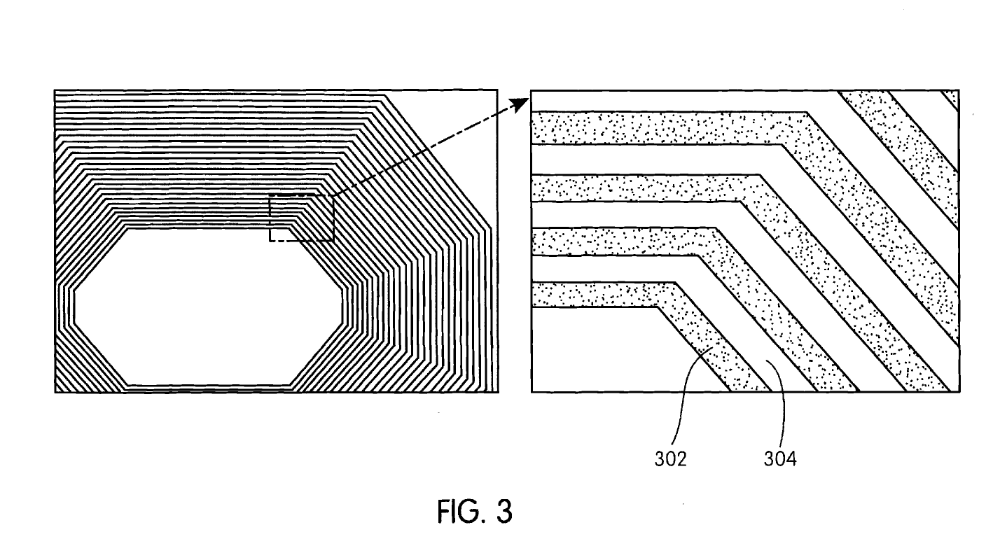

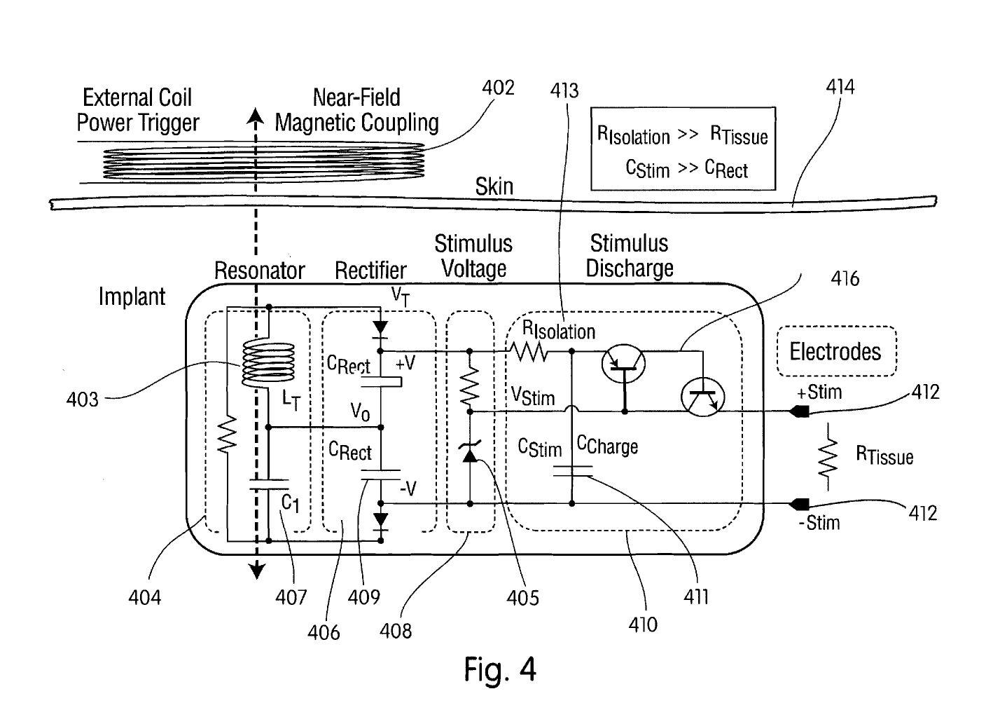

FIG. 4 is an illustration of a circuit diagram for a wireless microtransponder designed for independent auto-triggering operation (asynchronous stimulation), in accordance with one embodiment. As shown by the circuit diagram, the auto-triggering microtransponder includes a resonator element 404 (i.e., “tank circuit”), a rectifier element 406, a stimulus voltage element 408, a stimulus discharger element 410, and one or more electrodes 412. The resonator element 404 includes a coil (L_{T}) component 403 that is coupled to a capacitor (C_{T}) component 407. The resonator element 404 is configured to oscillate at a precise frequency that depends upon the values of these two components (i.e., the coil component 403 and capacitor component 407) as described in Equation 1:

Fᵣₑₛ=1/(2π√LC)

The resonator element 404 is coupled to the rectifier element 406, which is in turn coupled to the stimulus voltage element 408 and the stimulus discharger element 410. The rectifier element 406 and the stimulus voltage element 408 are both coupled in parallel to a capacitor 411. In addition, the stimulus discharger element 410 is coupled to electrodes 412, thereby electrically connecting the stimulus discharger element 410 to neural conduction tissue (axons). It should be appreciated that in certain embodiments, a voltage booster component (not shown) can be inserted immediately after the rectifier element 406 to boost the supply voltage available for stimulation and operation of integrated electronics beyond the limits generated by the miniaturized LC resonant ‘tank’ circuit 404. This voltage booster can enable electro-stimulation and other microtransponder operations using the smallest possible LC components which may generate too little voltage (<0.5V). Examples of high efficiency voltage boosters include charge pumps and switching boosters using low-threshold Schottky diodes. However, it should be understood that any type of conventional high efficiency voltage booster may be utilized in this capacity as long as it can generate the voltage required by the particular application of the microtransponder.

In this circuit configuration, the auto-triggering microtransponder can employ a bi-stable silicon switch 416 to oscillate between the charging phase that builds up a charge on the stimulus capacitor 411, and the discharge phase that can be triggered when the charge reaches the desired stimulation voltage by closing the switch 416 state to discharge the capacitor 411 through the stimulus electrodes 412. A single resistor 413 is used to regulate the stimulus frequency by limiting the charging rate. The breakdown voltage of a single zener diode 405 is configured to set the desired stimulus voltage by dumping current and triggering the switch 416 closure, discharging the capacitor 411 into the electrodes 412 (gold or Platinum-iridium alloy) when it reaches the stimulation voltage. Although gold was initially regarded as the preferred electrode material, it was discovered that in long-term implantation gold salt deposits could form and create a micro-battery, interfering with the stimulus signal. Gold remains a viable electrode material for some applications, but Platinum-iridium alloy is regarded as the preferred embodiment for long-term, permanent applications. Platinum is another acceptable electrode material.

The stimulus peak amplitude and duration are largely determined by the effective tissue (e.g., skin 414, muscle, fat etc.) resistance, independent of the applied RF power intensity. However, increasing the RF power may increase the stimulation frequency by reducing the time it takes to charge up to the stimulus voltage.

The auto-triggering microtransponder operates without timing signals from the RF power source (RF power coil) 402 and “auto-triggers” repetitive stimulation independently. As a result, the stimulation generated by a plurality of such auto-triggering microtransponders would be asynchronous in phase and somewhat variable in frequency from one stimulator to another depending upon the effective transponder voltage induced by each resonator circuit 404. While unique to this technology, there is no reason to predict that distributed asynchronous stimulation would be less effective than synchronous stimulation. In fact, such asynchronous stimulation may be more likely to evoke the sort of disordered “pins and needles” or “tingling” sensations of parasthesias that are associated with stimulation methods that most effectively block pain signals.

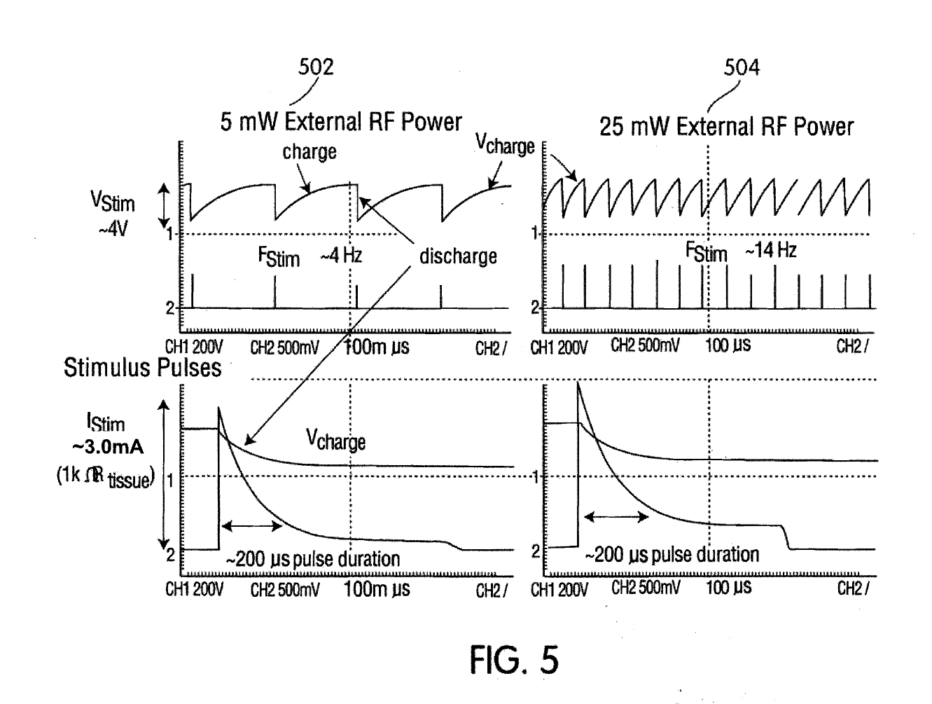

FIG. 5 presents several graphs that illustrate how wireless microtransponder stimulus frequencies, stimulus current peak amplitudes, and stimulus pulse durations vary under different device settings and external RF power input conditions, in accordance with one embodiment. In the first graph 502, the external RF power input is set at 5 mW resulting in a stimulus frequency of 4 Hz. As discussed previously, the stimulus frequency is a function of RF power as it directly affects the time it takes to charge up to the stimulus voltage. This direct relationship between RF power and stimulus frequency is clearly shown in graph 502 compared to graph 504, where the external RF power is ramped up from 5 mW to 25 mW, which results in a significant increase in stimulus frequency from 4 Hz to 14 Hz. It should be understood, however, that these are just examples of how RF power input settings affect stimulus frequency. In practice, the effects of the RF power input setting on stimulus frequency may be magnified or diminished depending on the particular application (e.g., depth of implantation, proximity to interfering body structures such as bones, organs, etc.) and device settings.

While RF intensity controls stimulus frequency, the stimulus voltage is typically controlled by the transponder zener diode element. The effect of stimulus voltage upon the stimulus current peak amplitude and pulse duration is further determined by the resistive properties of the tissue surrounding the microtransponder.

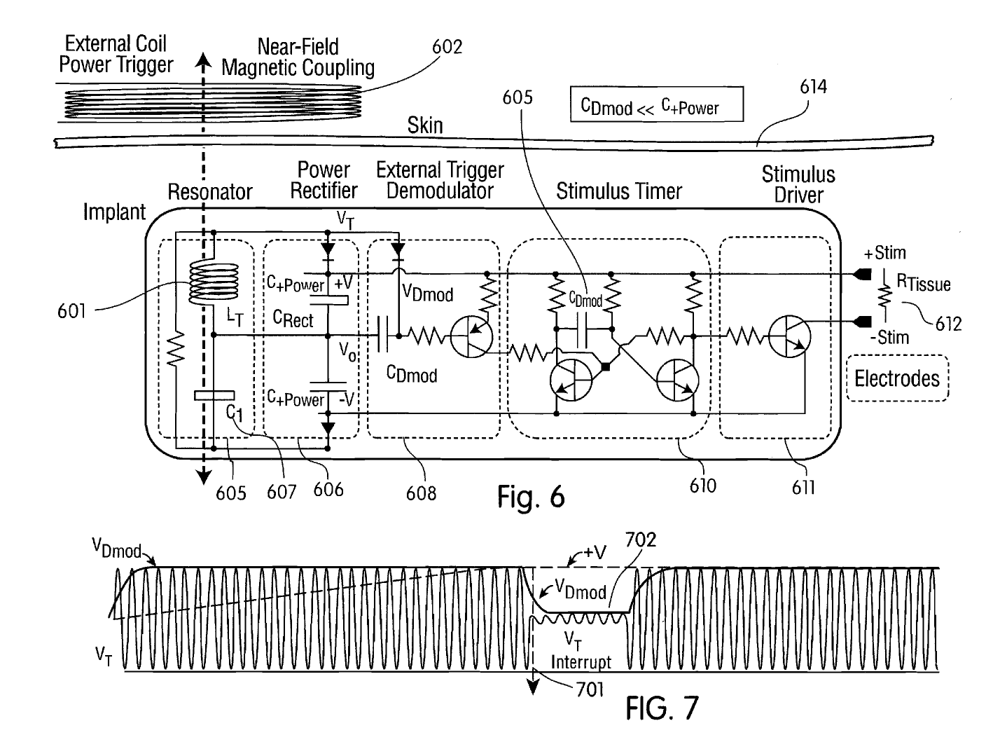

FIG. 6 is an illustration of a circuit diagram for a wireless microtransponder with an external trigger signal de-modulator element to synchronize the stimuli delivered with a plurality of other wireless microtransponders, in accordance with one embodiment. As depicted, herein, the wireless microtransponder design of FIG. 5 is modified to include an external trigger signal demodulator element 608 so that its' stimulus discharge can be synchronized by a trigger signal from an external RF power field.

The modified circuit includes a resonator element 604, a rectifier element 606, an external trigger demodulator element 608, a stimulus timer element 610, a stimulus driver element 611, and one or more electrodes 612. The resonator element 604 includes a coil (L_{T}) component 601 that is coupled to a capacitor (C_{T}) component 607. The resonator element 604 is configured to oscillate at a precise frequency that depends upon the values of these two components (i.e., the coil component 601 and capacitor component 607) as described in Equation 1.

The resonator element 604 is coupled to the rectifier element 606, which is in turn coupled to the external trigger demodulator element 608, the stimulus timer element 610, and the stimulus driver element 611. The rectifier element 606 and the stimulus timer element 608 are both coupled in parallel to the capacitor 607. In addition, the stimulus driver element 611 is coupled to electrodes 612 (gold or Platinum-iridium alloy), thereby electrically connecting the stimulus driver element 611 to neural conduction tissue (axons).

It should be appreciated that in certain embodiments, a voltage booster component (not shown) can be inserted immediately after the rectifier element 606 to boost the supply voltage available for stimulation and operation of integrated electronics beyond the limits generated by the miniaturized LC resonant ‘tank’ circuit (i.e. the coil component 601 and capacitor component 607). This voltage booster can enable electro-stimulation and other microtransponder operations using the smallest possible LC components which may generate too little voltage (<0.5V). Examples of high efficiency voltage boosters include charge pumps and switching boosters using low-threshold Schottky diodes. However, it should be understood that any type of conventional high efficiency voltage booster may be utilized in this capacity as long as it can generate the voltage required by the particular application that the microtransponder is applied to.

As shown in FIG. 7, the external synchronization-trigger circuit configuration (shown in FIG. 6) can employ a differential filtering method to separate the trigger signal, consisting of a sudden power interruption 701, from the slower drop in transponder power voltage 702 during the interruption. In particular, the circuit configuration (in FIG. 6) can utilize a separate capacitor (C_{Dur}) 605, in the stimulus timer element 610, to set the stimulus duration using a mono-stable multi-vibrator. Stimulus intensity can be controlled externally by the intensity of the applied RF power field generated by the external RF power coil 602. As the RF power field is modulated, the timing and frequency of stimuli from all the microtransponders under the external RF power coil 602 are synchronized externally.

Using the external synchronization-trigger circuit configuration (shown in FIG. 6), the degree of spatio-temporal control of complex stimulus patterns is essentially unlimited. In certain embodiments, the circuit configuration of the external synchronization-trigger circuit can be further modified so that it is configured to de-modulate the unique identity code of each microtransponder. This essentially permits the independent control of each microtransponder via RF signals. This added capability can provide a method to mediate the spatio-temporal dynamics necessary to restore natural sensations with artificial limbs or enable new sensory modalities (e.g., feeling infrared images, etc.).

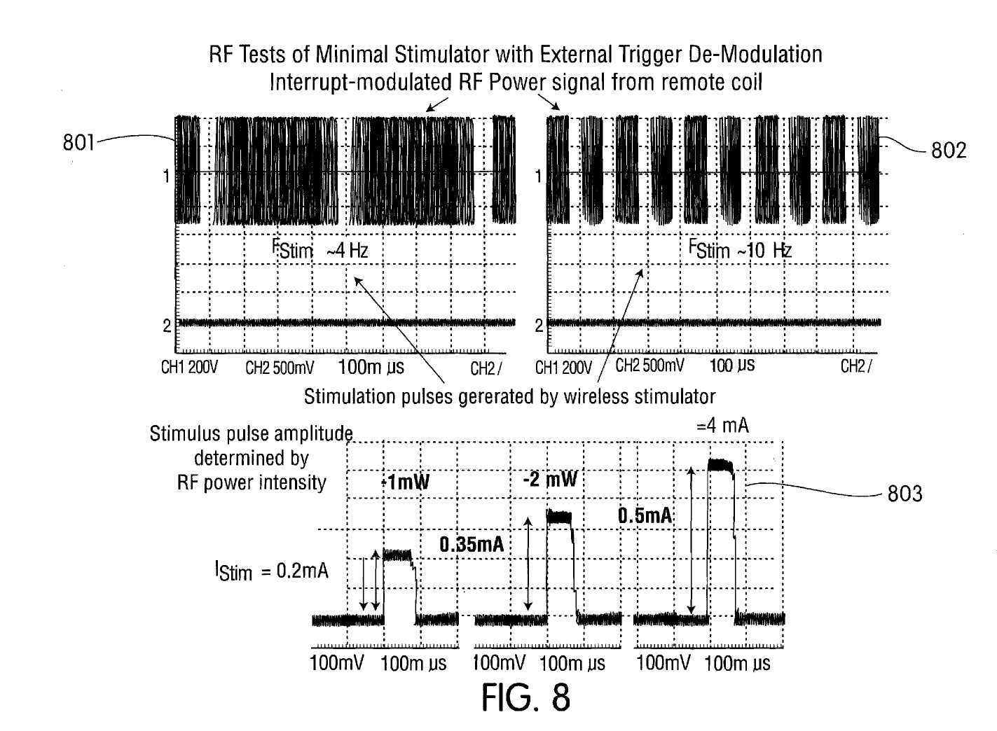

FIG. 8 presents several graphs that summarize the results from tests of a wireless microtransponder (with an external interrupt trigger de-modulator element) under different device settings and external RF power input conditions, in accordance with one embodiment. In the first graph 801, the external RF power coil modulates the RF power field to communicate a first trigger signal setting, which results in a stimulus frequency of 2 Hz. As discussed previously, the stimulus frequency is controlled by a trigger signal created when the RF power coil modulates the RF power field. The stimulus frequency is therefore directly related to the RF power field modulation frequency as shown in the second graph 802, where the stimulus frequency equals 10 Hz.

Whereas the stimulus frequency is controlled by external RF power field modulation settings, the stimulus current peak amplitude is controlled by the RF power intensity setting, as shown in the third graph 803. That is, the stimulus current peak amplitude is directly related to the RF power intensity setting. For example, an RF power intensity setting of 1 mW produces a stimulus current peak amplitude of 0.2 mA, a RF power intensity setting of 2 mW produces a stimulus current peak amplitude of 0.35 mA, and a RF power intensity setting of 4 mW produces a stimulus current peak amplitude of 0.5 mA. It should be understood, however, that these are just examples of how RF power intensity setting affects stimulus current peak amplitude. In practice, the effects of the RF power intensity setting on stimulus current peak amplitude may be magnified or diminished depending on the particular application (e.g., depth of implantation, proximity to interfering body structures such as bone, etc.) and device settings.

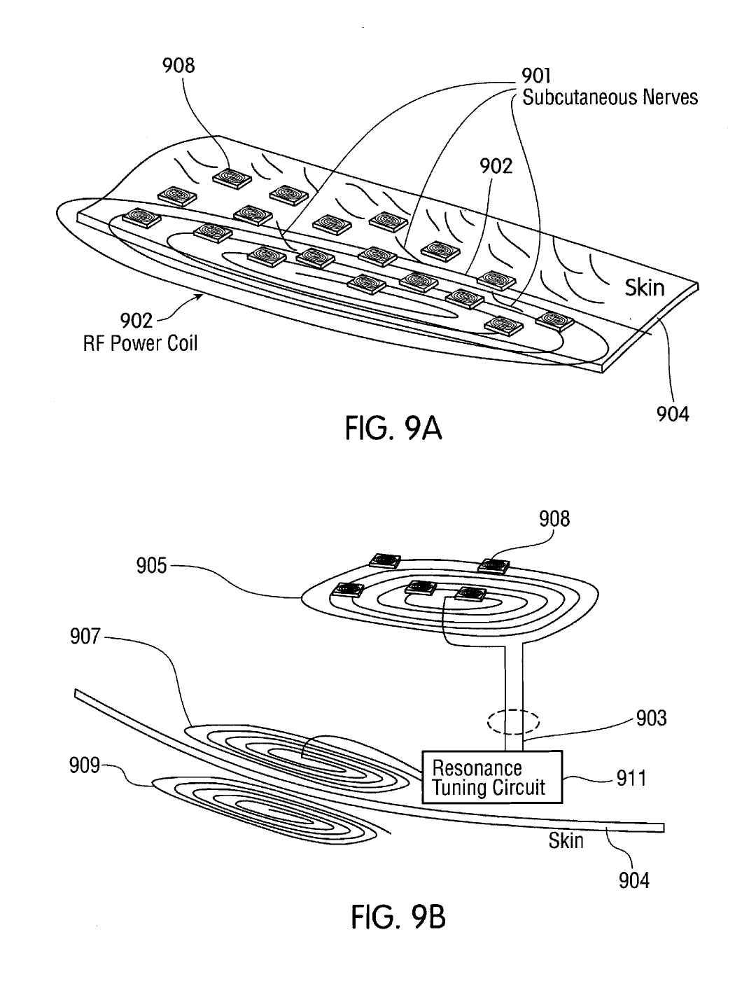

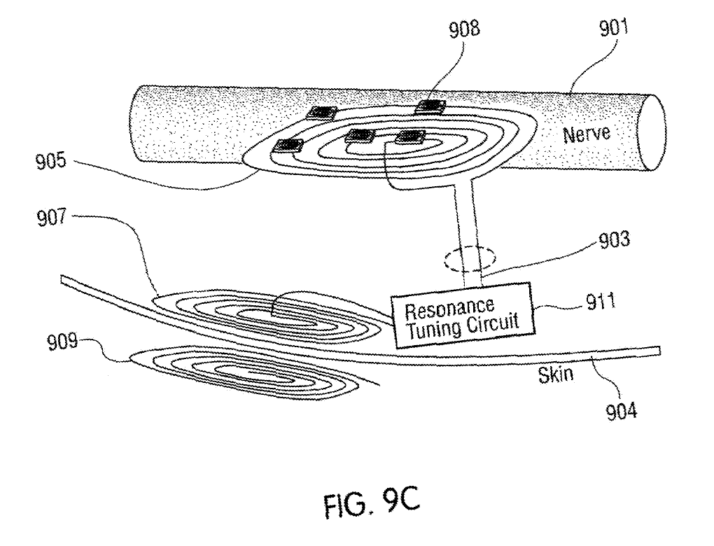

FIG. 9A is an illustration of a deployment of a plurality of wireless microtransponders distributed throughout subcutaneous vascular beds and terminal nerve fields, in accordance with one embodiment. As depicted, a plurality of independent wireless microtransponders 908 are implanted subcutaneously in a spread pattern under the skin 904 over the area that is affected. In this embodiment, each microtransponder is positioned proximate to and/or interfaced with a branch of the subcutaneous sensory nerves 901 to provide electrostimulation of those nerves. In one embodiment, only synchronous microtransponders are deployed. In another embodiment only asynchronous microtransponders are deployed. In yet another embodiment a combination of synchronous and asynchronous microtransponders are deployed.

After the deployment of the microtransponders, electrostimulation can be applied by positioning a RF power coil 902 proximate to the location where the microtransponders are implanted. The parameters for effective electrostimulation may depend upon several factors, including: the size of the nerve or nerve fiber being stimulated, the effective electrode/nerve interface contact, the conductivity of the tissue matrix, and the geometric configuration of the stimulating fields. While clinical and empirical studies have determined a general range of suitable electrical stimulation parameters for conventional electrode techniques, the parameters for micro-scale stimulation of widely distributed fields of sensory nerve fibers are likely to differ significantly with respect to both stimulus current intensities and the subjective sensory experience evoked by that stimulation.

Parameters for effective repetitive impulse stimulation using conventional electrode techniques are typically reported with amplitudes ranging to about 10 V (or up to about 1 mA) lasting up to about 1 millisecond repeated up to about 100 pulses/s for periods lasting several seconds to a few minutes at a time. In an exemplary embodiment, effective repetitive impulse stimulation can be achieved with an amplitude of less than 100 μA and stimulation pulses lasting less than 100 μs.

FIG. 9B is an illustration of a deployment of wireless microtransponders to enable coupling with deep microtransponder implants, in accordance with one embodiment. As shown herein, two simple electrical wires 903 lead from the subdermal/subcutaneous implanted outer transfer coil 907 to the deeper subcutaneous implanted inner transfer coil 903 proximate to a field of implanted micro-transponders 908. Threading the wires 903 through the interstitial spaces between muscles and skin involves routine minimally invasive surgical procedures as simple as passing the lead through hypodermic tubing, similar to routine endoscopic methods involving catheters. The minimal risks of such interstitial wires 903 are widely accepted.

The deep inner transfer coil 905 is implanted to couple with the deeply implanted field of micro-transponders 908 located near deep targets of micro-stimulation, such as deep peripheral nerves, muscles or organs such as the bladder or stomach as needed to treat a variety of clinical applications and biological conditions. The inner transfer coil 905 is tuned to extend the resonance of the external coil 909 to the immediate vicinity of the implanted micro-transponders 908 for maximal coupling efficiency. In addition to extending the effective range of the microtransponder 908 implants, the inner transfer coil 905 also provides another wireless link that can preserve the integrity of any further protective barrier around the target site. For instance, the inner transfer coil 905 can activate micro-transponders 908 embedded within a peripheral nerve without damaging the epineurium that protects the sensitive intraneural tissues. To ensure optimal tuning of the transfer coils (e.g., the outer transfer coil 907 and inner transfer coil 905), a variable capacitor or other tuning elements in a resonance tuning circuit 911 are added to the outer transfer coil 907 where it can be implanted with minimal risk of tissue damage. In certain embodiments, this resonance tuning circuit 911 is required, while in others it is unnecessary.

FIG. 9C is an illustration of a deployment of wireless microtransponders to enable coupling with deep neural microtransponder implants, in accordance with one embodiment. As shown herein, an extraneural inner transfer coil 905 positioned proximate to (or interfaced with) a nerve fiber or cell cluster 901 is interconnected to an outer transfer coil 907 by a simple pair of leads 903 that mediate all the signals and power necessary to operate micro-transponders 908 implanted anywhere in the body, beyond the direct effective range of powering by any external coil 909 (e.g., epidermal coil, etc.). In certain embodiments, the subdermal outer transfer coil 907 is tuned to the external coil 909 and implanted immediately under the external coil 909 just below the surface of the skin 904 for maximum near-field wireless magnetic coupling. This allows the RF waves generated by the external coil 909 to penetrate the body without long-term damage to the skin 904 and the risk of infection. In other embodiments, the outer transfer coil 907 is tuned to the external coil 909 and implanted deeper in the tissue subcutaneously. In some embodiments, a resonance tuning circuit 911 is required interposed between the inner transfer coil 905 and the outer transfer coil 907 to adjust the frequency of the signal at the inner transfer coil 905, while in others it is unnecessary.

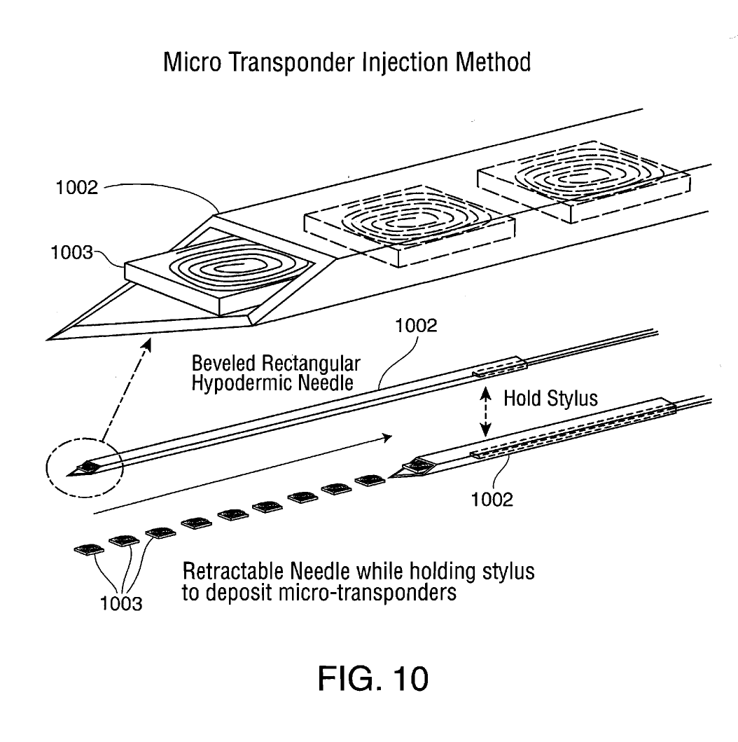

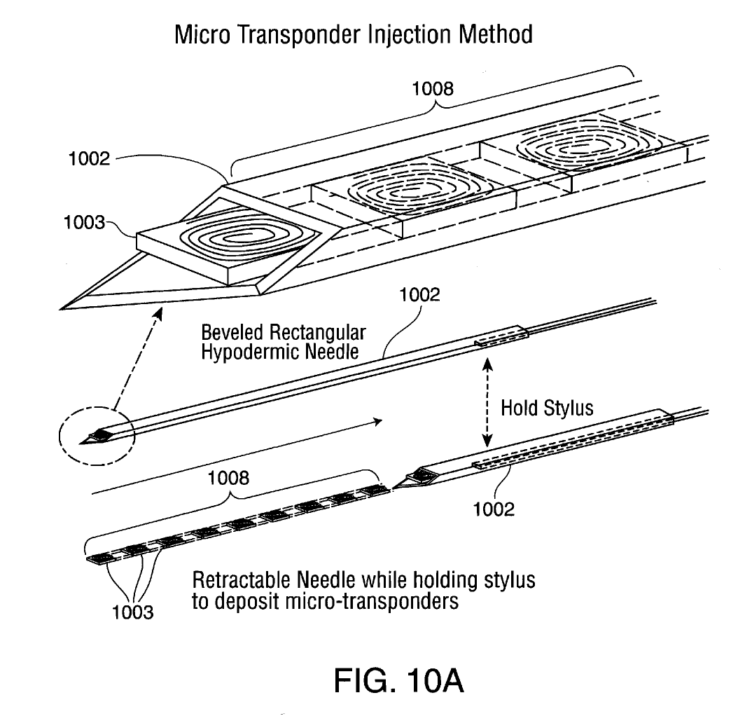

FIG. 10 is an illustration of how wireless microtransponders can be implanted using a beveled rectangular hypodermic needle, in accordance with one embodiment. As shown, the needle 1002 is curved to conform to the transverse cervical curvature (bevel concave) and without further dissection is passed transversely in the subcutaneous space across the base of the affected peripheral nerve tissue. Rapid insertion usually negates the need for even a short active general anesthetic once the surgeon becomes familiar with the technique. Following the placement of the microtransponders 1003 from the needle 1002, the needle 1002 is carefully withdrawn and the electrode placement and configuration is evaluated using intraoperative testing. Electrostimulation is applied using a temporary RF transmitter placed proximate to the location where the microtransponders 1003 are implanted, so the patient can report on the stimulation location, intensity, and overall sensation.

FIG. 10A is an illustration of how a joined array of wireless microtransponders can be implanted using a beveled rectangular hypodermic needle, in accordance with one embodiment. As in FIG. 10, the needle 1002 is curved to conform to the transverse cervical curvature (bevel concave) and without further dissection is passed transversely in the subcutaneous space across the base of the affected peripheral nerve tissue with rapid insertion usually negating the need for any anesthetic. The microtransponders 1003 are joined together to form a joined array 1008.

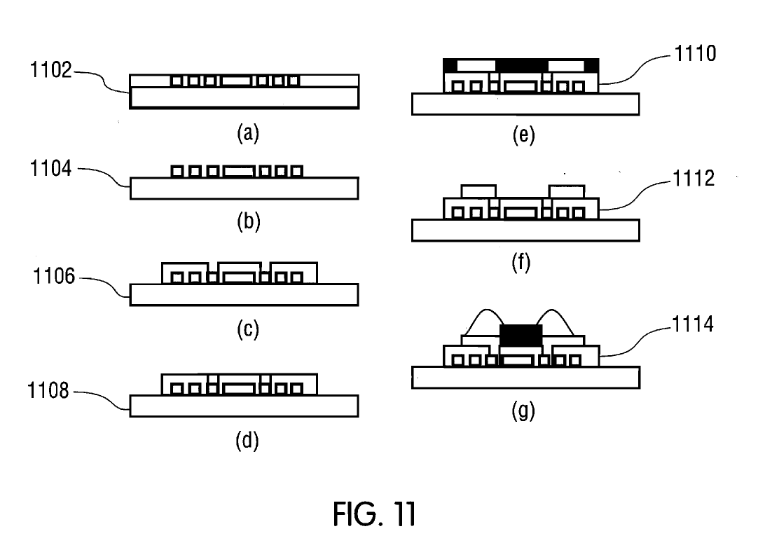

FIG. 11 is an illustration of a fabrication sequence for spiral type wireless microtransponders, in accordance with one embodiment. At step 1102, a layer of gold spiral coil is electroplated onto a substrate (typically a Pyrex® based material, but other materials may also be used as long as they are compatible with the conducting material used for the spiral coil and the particular application that the resulting microtransponder will be applied to). Electroplated gold is used as the conductor material due to its high conductivity, resistance to oxidation, and proven ability to be implanted in biological tissue for long periods of time. It should be appreciated, however, that other conducting materials can also be used as long as the material exhibits the conductivity and oxidation resistance characteristics required by the particular application that the microtransponders would be applied to. Typically, the gold spiral coil conductors have a thickness of between approximately 5 μm to approximately 25 μm.

In one embodiment, the gold spiral coil takes on a first configuration where the gold conductor is approximately 10 μm wide and there is approximately 10 μm spacing between the windings. In another embodiment, the gold spiral coil takes on a second configuration where the gold conductor is approximately 20 μm wide and there is approximately 20 μm spacing between the windings. As will be apparent to one of ordinary skill in the art, however, the scope of the present invention is not limited to just these example gold spiral coil configurations, but rather encompasses any combination of conductor widths and winding spacing that are appropriate for the particular application that the coil is applied to.

In step 1104, the first layer of photoresist and the seed layer are removed. In one embodiment, the photoresist layer is removed using a conventional liquid resist stripper to chemically alter the photoresist so that it no longer adheres to the substrate. In another embodiment, the photoresist is removed using a plasma ashing process.

In step 1106, an isolation layer of SU-8 photo resist is spun and patterned to entirely cover each spiral inductor. Typically, the SU-8 layer has a thickness of approximately 30 μm. In step 1108, a top seed layer is deposited on top of the SU-8 isolation layer using a conventional physical vapor deposition (PVD) process such as sputtering. In step 1110, a top layer of positive photo resist coating is patterned onto the top see layer and the SU-8 isolation layer, and in step 1112, a layer of platinum is applied using a conventional electroplating process. In step 1114, a chip capacitor and a RFID chip are attached to the platinum conducting layer using epoxy and making electrical connections by wire bonding. In certain embodiments, the capacitor has a capacitance rating value of up to 10,000 picofarad (pF).

It is possible to implant such small microtransponders by simply injecting them into the subdermal tissue. Using local anesthesia at the injection site, the patient may be positioned laterally or prone depending on the incision entry point. The subdermal tissues immediately lateral to the incision are undermined sharply to accept a loop of electrode created after placement and tunneling to prevent electrode migration. A Tuohy needle is gently curved to conform to the transverse posterior cervical curvature (bevel concave) and without further dissection is passed transversely in the subdermal space across the base of the affected peripheral nerves. Rapid needle insertion usually obviates the need for even a short acting general anesthetic once the surgeon becomes facile with the technique. Following placement of the electrode into the Tuohy needle, the needle is withdrawn and the electrode placement and configuration is evaluated using intraoperative testing.

After lead placement, stimulation is applied using a temporary RF transmitter to various select electrode combinations enabling the patient to report on the table the stimulation location, intensity and overall sensation. Based on prior experience with wired transponders, most patients should report an immediate stimulation in the selected peripheral nerve distribution with voltage settings from 1 to 4 volts with midrange pulse widths and frequencies. A report of burning pain or muscle pulling should alert the surgeon the electrode is probably placed either too close to the fascia or intramuscularly.

An exemplary microtransponder array preferably is an array of joined microtransponders. The joined array is made from or coated with biocompatible material that is sufficiently strong to hold the microtransponders and remain intact during surgical explantation. An advantage of the joined array is that removal of the array is simpler than unjoined microtransponders, which would be more difficult to locate and individually extract from the integrated mass of adhered tissues. The concept is flexible, as the array may comprise a joined array of any type of implanted medical devices. The monolithic array structure cam hold the implanted devices together during explantation.

The joined array can be made from several types of biocompatible materials. Exemplary synthetic materials suitable for the removable array include silicone elastomers, or silicone hydrogels, and plastics such as SU-8, or parylene-C. Removable arrays may also be constructed using long-lasting biodegradable polymers including natural materials such as protein-based polymers like gelatin, silk or collagen, and sugar-based poly-saccharides like cellulose or agarose. Other suitable biodegradable polymers have been developed specifically for implant construction including poly-glyolic acids (PGA) and poly-lactic acids (PLA). Such construction materials offer a range of strengths, durability and tissue adhesion properties suitable for a variety of specific implant applications. Furthermore, the surface of any array material may be enhanced to promote specific biological properties such as cell/protein adhesion and tissue reactions by coating the implant with a variety of materials widely employed for this purpose including formulations of PEG (polyethylene glycol) such as PEG-PLA, and commercial products such as Greatbatch Biomimetic Coating (U.S. Pat. No. 6,759,388 B1), and Medtronics' Trillium Biosurface.

Biocompatibility of the array is very important. The joined array can include a coating in the form of a monolayer or thin layer of biocompatible material. Advantages that coatings offer include the ability to link proteins to the coating. The joined proteins can limit what cell types can adhere to the array. The coating can prevent protein adsorption, and it does not significantly increase size of the device.

3-D porous materials are meant to encourage cell ingrowth and organization. The 3-D porous material can act as a buffer between the tissue and microtransponders to prevent reaction micromotion. The potential benefits for implant/tissue integration must be balanced against the addition risks associated with increasing the overall size of the implant with the addition of such 3-D materials.

The visibility of the implant may be enhanced by adding brightly colored dyes to the construction materials thereby facilitating visual location of the array within surrounding tissue in case it must be removed. This can include a marker dye incorporated onto, or into, the device globally. A preferred embodiment would employ a fluorescent dye that becomes visible when exposed to appropriate light sources because it offers the advantage of maximum luminescence to such a level that implants may be visible through the skin.

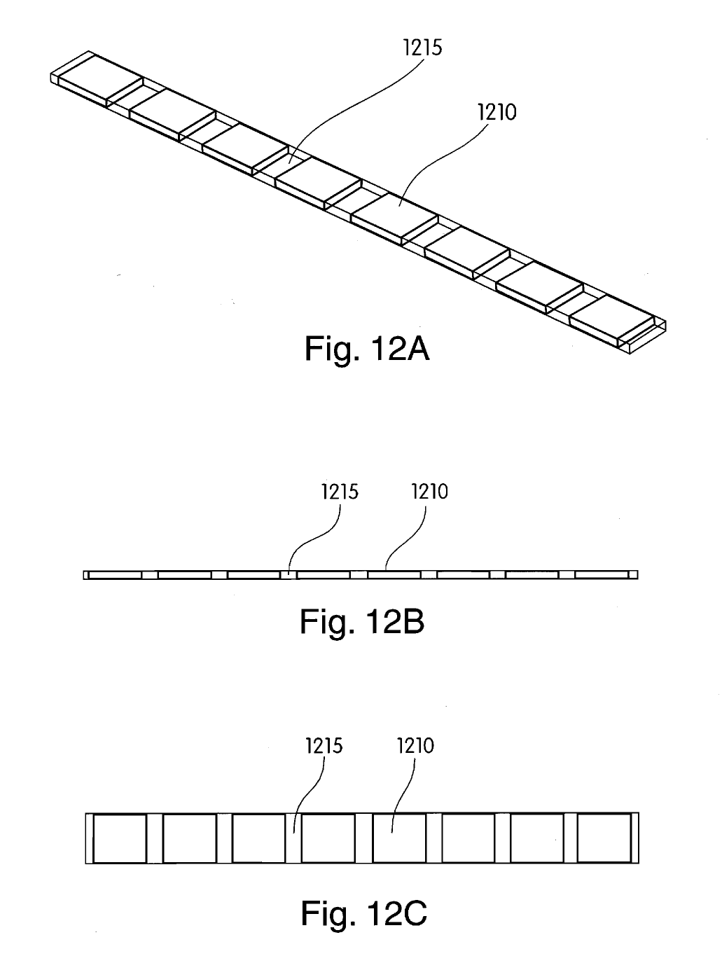

FIG. 12A shows a perspective view of the basic embodiment of an array. The joined array 1215 comprises a prefabricated strip where each microtransponder 1210 is joined to adjacent microtransponders 1210 using SU-8 to conserve continuity. FIG. 12B shows a side view of the basic embodiment of an array. The array 1215 is composed of SU-8 and joined microtransponders 1210. FIG. 12C shows an overhead view of the basic embodiment of an array. An advantage of this design is that no extra materials or steps are required for production of the solid joined array, making it relatively simple to fabricate.

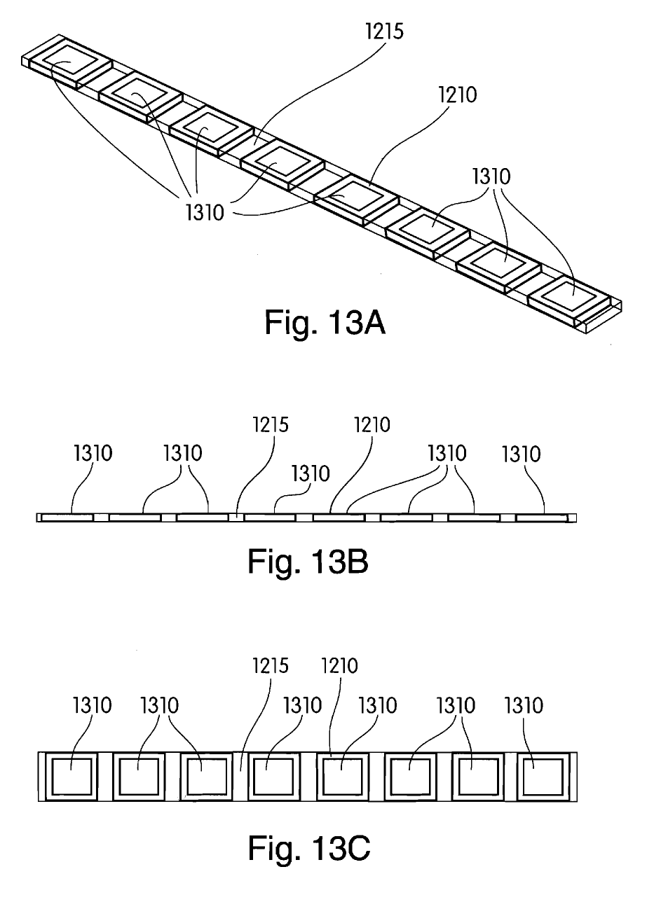

FIG. 13A shows a perspective view of an array comprising exposed electrodes through windows in the array. The joined array 1215 comprises a strong strip containing a joined array of individual microtransponders 1210, where each microtransponder 1210 is joined to adjacent microtransponders 1210. In this embodiment, the superior and inferior electrodes are exposed through windows 1310 in the microtransponders 1210. FIG. 13B shows a side view of an array 1215 comprising exposed electrodes through windows 1310 in the array 1215. FIG. 13C shows an overhead view of an array comprising exposed electrodes through windows 1310 in the array 1215. This embodiment can use a more durable material than SU-8 and the joined and embedded microtransponders are better protected. Additionally, the array can be more flexible than a prefabricated SU-8 solid array.

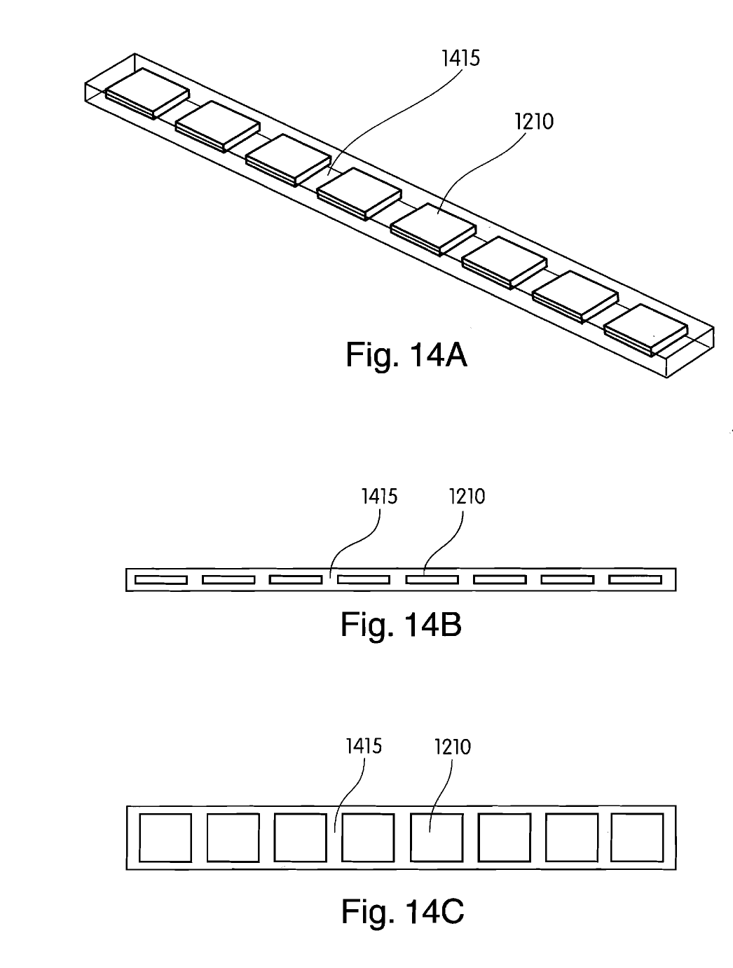

FIG. 14A shows a perspective view of an array comprising an ion permeable strip. The ion permeable joined array 1415 resists ingrowth of surrounding tissue, and the joined individual microtransponders 1210 are totally embedded within the array 1415. FIG. 14B shows a side view of an ion permeable array. The microtransponders 1210 are totally embedded within the ion permeable array 1415. FIG. 14C shows an overhead view of an array comprising an ion permeable strip. This embodiment can use a more durable material than SU-8 and the embedded microtransponders 1210 are better protected. Additionally, the array 1415 can be more flexible than a prefabricated SU-8 solid array. The electrodes can be totally isolated from proteins and tissues, but still affect ions in solution. There is possible reduced efficacy as tissue would be kept a minimum distance away from electrodes.

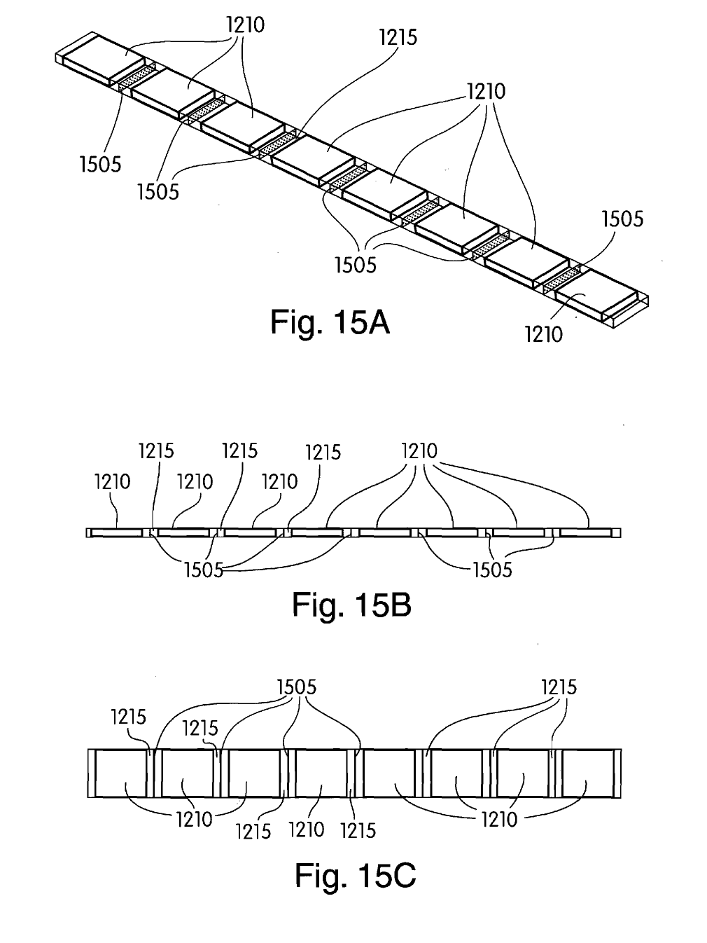

FIG. 15A shows a perspective view of the basic embodiment of a slotted array. This type of array is intended for permanent implantation and includes a slot depressed into the surface or entirely through sites along the array or in the micro-transponders themselves intended for tissue ingrowth to secure the array in place. The joined slotted array 1215 comprises a prefabricated strip where each microtransponder 1210 is joined to adjacent microtransponders 1210 using SU-8 to conserve continuity. Portions of the array surface, such as directly over the microtransponders 1210, can be coated with a material to prevent protein adsorption. Slots 1505 through the array 1215 between the microtransponders are intended to receive tissue ingrowth to permanently anchor the array 1215 in place. FIG. 15B shows a side view of the basic embodiment of a slotted array. The array 1215 comprises SU-8 and joined microtransponders 1210 with slots 1505 passing through the array 1215. FIG. 15C shows an overhead view of the basic embodiment of a slotted array.

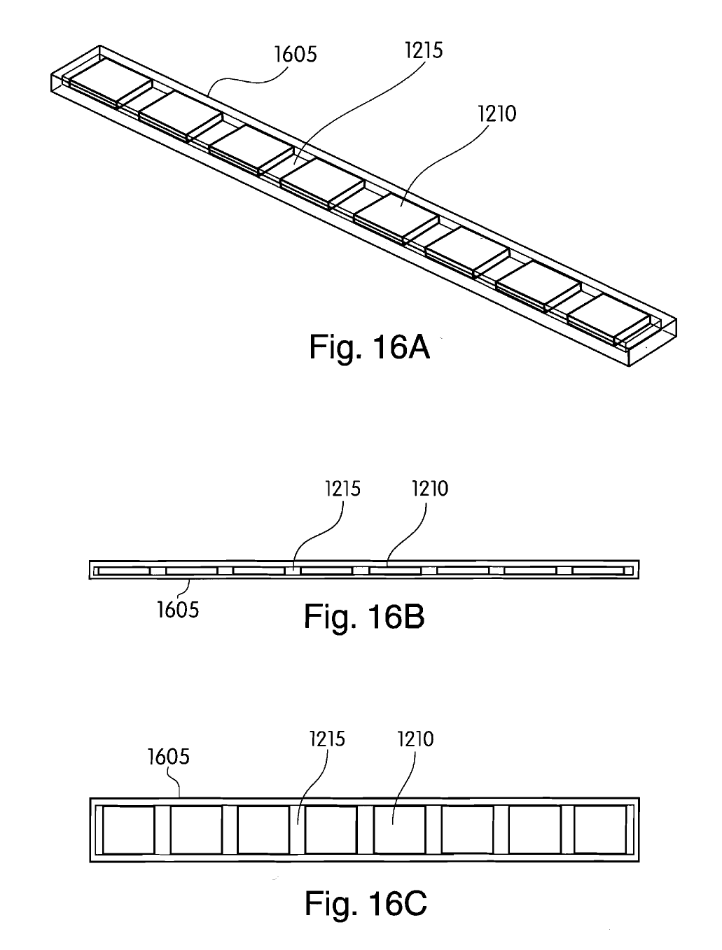

FIG. 16A shows a perspective view of the basic embodiment of an array surrounded by an enveloping matrix. The joined array 1215 comprises a prefabricated strip where each microtransponder 1210 is joined to adjacent microtransponders 1210 using SU-8 to conserve continuity. A matrix 1605 of biocompatible material surrounds the joined array 1215 to fully surround the joined array 1215. FIG. 16B shows a side view of the basic embodiment of an array surrounded by an enveloping matrix. The biocompatible matrix 1605 encases the joined array 1215 of joined microtransponders 1210. FIG. 12C shows an overhead view of the basic embodiment of an array surrounded by an enveloping matrix. An advantage of this design is that no extra materials or steps are required for production of the joined array 1215, making it relatively simple to fabricate and encase in the matrix.

The joined array can also be formed from a biological degradable material. As the joined array material dissolved, the microtransponders would be freed to move freely and minimize tissue reactions. The most common examples of biodegradable materials include natural polymers based on proteins (e.g. gelatin, collagen, silk) and poly-saccharides (sugar-based polymers like cellulose and starch), in various formulations (i.e. proteo-saccharides like agarose) that provide a wide range of strength and degradation times. Other known acceptable biodegradable materials include polyglycolic acid (PGA) and polylactic acid (PLA).

Of course, the innovations of the present application are not limited to the embodiments disclosed, but can include various materials, configurations, positions, or other modifications beyond these embodiments shown, which are exemplary only.

According to various embodiments, there is provided: a microtransponder array, comprising: an array comprised of adjacent and physically joined wireless microtransponders; wherein each microtransponder is wirelessly interfaced.

According to various embodiments, there is provided: an implantable device, comprising: an array of physically joined embedded wirelessly interfaced microtransponders; wherein electrode surfaces on the array are exposed by windows in the individual microtransponders.

According to various embodiments, there is provided: a method of forming an implantable wireless electronic device, comprising the steps of: creating a removable array of embedded adjacent electronic components on a single substrate; and powering the array using a wireless interface.

According to various embodiments, there is provided: a method for implanting wireless electronics into living tissue, comprising: implanting an array of physically joined and wirelessly powered electronic devices into tissue; and if removal of the electronic devices is necessary, then exposing the array of joined electronic device, and thereafter removing the array of electronic devices from the living tissue.

According to various embodiments, there is provided: an electronic device for implantation, comprising: an array of physically joined embedded wireless components; wherein the array is ion permeable and resists ingrowth of nonconductive fibrous matter.

According to various embodiments, there is provided: a method of removing an implanted plurality of electronic devices, comprising the steps of: implanting the array with a surrounding matrix; keeping tissue growth a minimum distance away from at least a portion of the joined electronic devices; locating the array using an incorporated mark; and surgically exposing the array to grasp and pull free.

According to various embodiments, there is provided: a biocompatible electronic module implantable into living tissue, comprising: a plurality of electronic devices wirelessly powered and coupled together to form a physically joined array of a size permitting implanting from a needle; and at least one electrical conduction path through said array that connects at least one terminal of said device to surrounding tissue.

### MODIFICATIONS AND VARIATIONS

As will be recognized by those skilled in the art, the innovative concepts described in the present application can be modified and varied over a tremendous range of applications, and accordingly the scope of patented subject matter is not limited by any of the specific exemplary teachings given.

For example, in one embodiment, rather than an elongated or linear strip, the joined microtransponders can be joined both longitudinally and latitudinally to form a geometric shape. The shapes can include squares, hexagons, rectangles, ovals, and circles.

The array can also be formed on a single substrate, with a chain or group of arrays constructed contemporaneously to form a single integrated structure. It may also be possible to construct joined arrays using a monofilament line as a string of microtransponders.

The specific implementations given herein are not intended to limit the practice of the present innovations.

The following applications may contain additional information and alternative modifications: Attorney Docket No. MTSP-29P, Ser. No. 61/088,099 filed Aug. 12, 2008 and entitled “In Vivo Tests of Switched-Capacitor Neural Stimulation for Use in Minimally-Invasive Wireless Implants; Attorney Docket No. MTSP-30P, Ser. No. 61/088,774 filed Aug. 15, 2008 and entitled “Micro-Coils to Remotely Power Minimally Invasive Microtransponders in Deep Subcutaneous Applications”; Attorney Docket No. MTSP-31P, Ser. No. 61/079,905 filed Jul. 8, 2008 and entitled “Microtransponders with Identified Reply for Subcutaneous Applications”; Attorney Docket No. MTSP-33P, Ser. No. 61/089,179 filed and entitled “Addressable Micro-Transponders for Subcutaneous Applications”; Attorney Docket No. MTSP-36P Ser. No. 61/079,004 filed Jul. 8, 2008 and entitled “Microtransponder Array with Biocompatible Scaffold”; Attorney Docket No. MTSP-38P Ser. No. 61/083,290 filed Jul. 24, 2008 and entitled “Minimally Invasive Microtransponders for Subcutaneous Applications” Attorney Docket No. MTSP-39P Ser. No. 61/086,116 filed Aug. 4, 2008 and entitled “Tintinnitus Treatment Methods and Apparatus”; Attorney Docket No. MTSP-40P, Ser. No. 61/086,309 filed Aug. 5, 2008 and entitled “Wireless Neurostimulators for Refractory Chronic Pain”; Attorney Docket No. MTSP-41P, Ser. No. 61/086,314 filed Aug. 5, 2008 and entitled “Use of Wireless Microstimulators for Orofacial Pain”; Attorney Docket No. MTSP-42P, Ser. No. 61/090,408 filed Aug. 20, 2008 and entitled “Update: In Vivo Tests of Switched-Capacitor Neural Stimulation for Use in Minimally-Invasive Wireless Implants”; Attorney Docket No. MTSP-43P, Ser. No. 61/091,908 filed Aug. 26, 2008 and entitled “Update: Minimally Invasive Microtransponders for Subcutaneous Applications”; Attorney Docket No. MTSP-44P, Ser. No. 61/094,086 filed Sep. 4, 2008 and entitled “Microtransponder MicroStim System and Method”; Attorney Docket No. MTSP-30, Ser. No. ______, filed ______ and entitled “Transfer Coil Architecture”; Attorney Docket No. MTSP-31, Ser. No. ______, filed ______ and entitled “Implantable Driver with Charge Balancing”; Attorney Docket No. MTSP-32, Ser. No. ______, filed ______ and entitled “A Biodelivery System for Microtransponder Array”; Attorney Docket No. MTSP-46, Ser. No. ______, filed ______ and entitled “Implanted Driver with Resistive Charge Balancing”; Attorney Docket No. MTSP-28, Ser. No. ______, filed ______ and entitled “Implantable Transponder Systems and Methods”; and Attorney Docket No. MTSP-48, Ser. No. ______ filed ______ and entitled “Implantable Transponder Pulse Stimulation Systems and Methods” and all of which are incorporated by reference herein.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

1. A microtransponder array, comprising:
an array comprised of adjacent and physically joined wireless microtransponders;
wherein each microtransponder is wirelessly interfaced.

2. (canceled)

3. The array of claim 1, wherein the array includes both longitudinal and latitudinal joined wireless microtransponders.

4. (canceled)

5. The array of claim 1, wherein the joined wireless microtransponders form a geometric shape that can include at least one of the following:
elongated strip;
square;
rectangle;
hexagon;
circle; and
oval.

6. The array of claim 1, wherein the array comprises a material selected from the following group:
silicone elastomers;
silicone hydrogels;
plastic.

7. (canceled)

8. The array of claim 1, wherein the array is biodegradable and comprises a material selected from the following group:
protein-based polymers;
sugar-based poly-saccharides;
poly-glyolic acids (PGA); and
poly-lactic acids (PLA).

9-10. (canceled)

11. The array of claim 1, wherein the array is coated with material that comprises a material selected from the following group:
polyethylene glycol (PEG);
poly-lactic acids (PLA);
biomimetic coating; and
trillium biosurface.

12. (canceled)

13. The array of claim 1, wherein the wireless microtransponders expose superior and inferior electrodes through windows.

14. The array of claim 1, further comprising:
an ion permeable material that resist ingrowth of surrounding tissue; and
joined wireless microtransponders totally embedded within.

15-47. (canceled)

48. An electronic device for implantation, comprising:
an array of physically joined embedded wireless components;
wherein the array is ion permeable and resists ingrowth of nonconductive fibrous matter.

49. The electronic device of claim 48, wherein the array is marked.

50. The electronic device of claim 49, wherein the marking can include a fluorescent dye marker visible under appropriate light sources through the skin.

51. The method of claim 48, wherein the array includes both longitudinal and latitudinal joined wireless components.

52. The method of claim 48, wherein the array of joined embedded components is removable and coated with a material selected from the following group:
polyethylene glycol (PEG);
poly-lactic acids (PLA);
biomimetic coating; and
trillium biosurface.

53. The array of claim 48, wherein the array comprises a biological degradable material.

54-63. (canceled)

64. A biocompatible electronic module implantable into living tissue, comprising:
a plurality of electronic devices wirelessly powered and coupled together to form a physically joined array of a size permitting implanting from a needle; and
at least one electrical conduction path through said array that connects at least one terminal of said device to surrounding tissue.

65. (canceled)

66. The biocompatible electronic module of claim 64, wherein the array is physically joined using material selected from the following group:
silicone elastomers;
silicone hydrogels;
plastic; and
monofilament.

67. The biocompatible electronic module of claim 64, wherein the electronic device is coated with material that comprises a material selected from the following group:
polyethylene glycol (PEG);
poly-lactic acids (PLA);
biomimetic coating; and
trillium biosurface.

68-69. (canceled)

70. The biocompatible electronic module of claim 64, wherein the array comprises an ion permeable strip that resists ingrowth of surrounding tissue and joined microtransponders that are totally embedded within.

71. The biocompatible electronic module of claim 64, wherein the array keeps tissue a minimum distance away from at least a portion of the electronic device.

72. The biocompatible electronic module of claim 64, wherein the electronic device includes a single substrate structure.

73. (canceled)

74. The biocompatible electronic module of claim 64, wherein the array comprises a biological degradable material.
